(19) United States
(12) Patent Application Publication
(10) Pub. No.: US 2007/0044681 A1
(43) Pub. Date: Mar. 1, 2007

(54) METHOD FOR MANUFACTURING MONASCUS PIGMENT

(76) Inventors: Naoko Tsuyoshi, Kawasaki-shi (JP); Tomomi Kuwahara, Kawasaki-shi (JP); Ryosuke Fudou, Kawasaki-shi (JP); Toshimi Mizukoshi, Kawasaki-shi (JP); Hiroo Yoshida, Kawasaki-shi (JP)

Correspondence Address: AJINOMOTO CORPORATE SERVICES LLC, 1120 CONNECTICUT AVENUE, N.W., SUITE 1010, WASHINGTON, DC, 20036, US

(21) Appl. No.: 11/468,382

(22) Filed: Aug. 30, 2006

(30) Foreign Application Priority Data
Sep. 1, 2005 (JP) 2005-253432

(51) Int. Cl. C12P 19/28; C12N 1/16; C09D 11/00

(52) U.S. Cl. 106/31.68; 435/85; 536/17.4; 435/254.1

(57) ABSTRACT

A compound denoted by general formula (1) below; 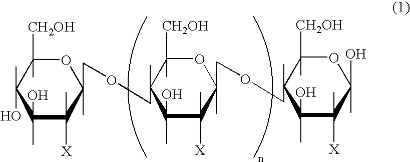
wherein n denotes an integer from 0 to 8 and wherein each X denotes either a residue denoted by general formula (2) below or an amino group and at least one X is a residue denoted by general formula (2) below; and 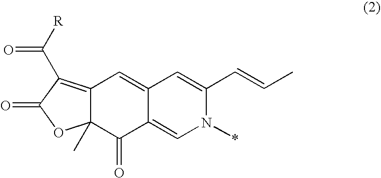
wherein R denotes C₅H₁₁ or C₇H₁₅ is provided.

This application claims priority under 35 U.S.C. §119(a) to JP 2005-253432, filed in Japan on Sep. 1, 2005, the entirety of which is incorporated by reference.

### BACKGROUND OF THE INVENTION

1. Field of the Invention

The present invention relates to monascus pigments, which are natural red pigments primarily employed to color foods, cosmetics, and the like, and more particularly, to a monascus pigment with enhanced stability, particularly in aqueous solutions.

2. Brief Description of the Related Art

Monascus pigments are pigments produced by filamentous fungi (monascus fungi) of the genus Monascus. They have been used since ancient times in China, Taiwan, and the like as colorants in red alcohol beverages, meat, and the like. Their safety has been confirmed. Generally, the monascus pigments have a composition comprised of compounds of similar structure but different substituents, such as the orange-colored monascorubrin, orange-colored rubropunctatin, yellow-colored ankaflavin, yellow-colored monascin, red-colored monascorubramin, and red-colored rubropunctamine (J. Ferment, Technol., Vol. 51, p, 407 (1973)). These compounds are insoluble in water, but monascorubrin and rubropunctatin react in the culture solution with water-soluble compounds having an amino group, such as water-soluble proteins, peptides, and amino acids, to form water-soluble complexes that are referred to as red-colored water-soluble monascus pigments (Journal of Industrial Microbiology, Vol. 16, pp. 163-170 (1996)). However, the monascus pigments are somewhat unstable with respect to light and heat. Improvement is required so that they do not discolor or fade. One known method of preventing the fading of monascus pigments is to store them in butanol of ethanol. However, many monascus pigments are employed in products containing water, precluding use of this method.

Other known methods of preventing lading involve adding tea extract, myrica extract, sesamol, vitamin C, or the like to monascus pigments (Japanese Patent Application Publication No. 2002-173609, Japanese Patent Application Publication No. Hei 6-2.34935, and Japanese Patent Application Publication No. Hei 10-110109; Nutrition and Food, Vol. 28, No. 4, pp. 207-211 (1975)). However, none of these methods is satisfactory at present.

### SUMMARY OF THE INVENTION

Based on these problems, the present invention has for its object to provide a water-soluble monascus pigment with improved stability in aqueous solutions.

The present inventors conducted extensive research into solving the above-stated problems, resulting in the discovery that when a compound having an amino group in the form of a chitosan oligosaccharide was bonded to a pigment produced by a monascus fungus in the form of rubropunctatin or monascorubrin, the resulting water-soluble red pigment was extremely stable in aqueous solutions. A method for manufacturing a colorant in which the compound having an amino group is glucosamine or a polymer thereof in the form of chitosan is already known (Japanese Patent Publication No. Sho 52-32965), Based on investigation by the present inventors, when the compound having an amino group was the monosaccharide glucosamine, stability did not improve, and when chitosan, water solubility was extremely poor, falling short of the mark. Conversely, when a chitosan oligosaccharide (with a degree of polymerization of 2 to 10) was employed as the compound having an amino group, the present inventors discovered that stability improved and it became possible to manufacture a pigment that was highly soluble in water.

That is, the chitosan oligosaccharide-bonded pigment of the present invention is characterized by comprising a compound having a chitosan oligosaccharide residue as a principal component of a pigment, and contains the followings.

It is an object of the present invention to provide a compound denoted by general formula (1) below;

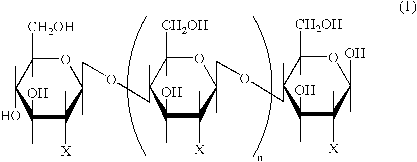

wherein n denotes an integer from 0 to 8 and wherein each X denotes either a residue denoted by general formula (2) below or an amino group and at least one X is a residue denoted by general formula (2) below; and 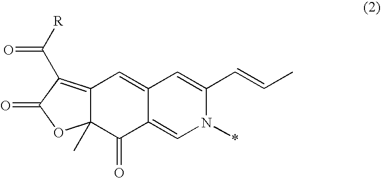
wherein R denotes C₅H₁₁ or C₇H₁₅

It is an object of the present invention to provide the compound described above, which is denoted by general formula (3) below; 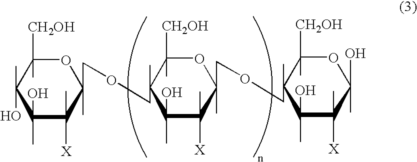
wherein n denotes an integer from 0 to 8 and wherein one X is a residue denoted by general formula (2) below and the others are amino groups; and 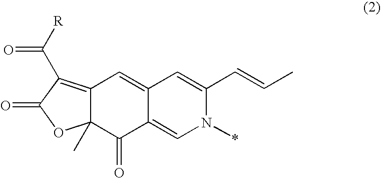
wherein R denotes C₅H₁₁ or C₇H₁₅.

It is an object of the present invention to provide a water-soluble composition comprising a mixture of one or more of the compounds described above.

It is an object of the present invention to provide a pigment comprising one or more of the compounds described above.

It is an object of the present invention to provide a pigment composition comprising a mixture of one or more of the compounds described above and an ink-use solvent.

It is an object of the present invention to provide a method for manufacturing the compound described above comprising at least the steps of:

supplying a chitosan oligosaccharide denoted by general formula (4) below and a compound denoted by general formula (5) below to the transamination reaction denoted by reaction equation (6) below at pH of between 6 and 10; and

replacing the oxygen atom of a pyrane ring in the compound denoted by general formula (5) below with the nitrogen atom of an amino group on at least one structural sugar in the oligosaccharide; 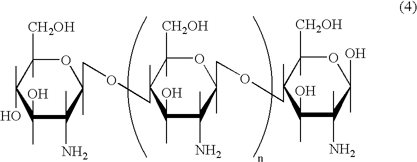
wherein n denotes an integer from 0 to 8; 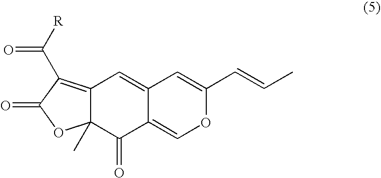
wherein R denotes C₅H₁₁ or C₇H₁₅. 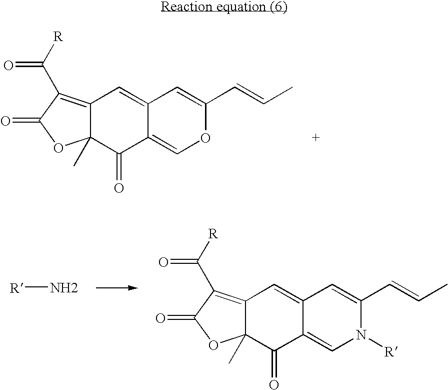
wherein R denotes C₅H₁₁ or C₇H₁₅ and R′ denotes a side chain moiety other than an amino group of the chitosan oligosaccharide.

It is an object of the present invention to provide the method described above, wherein the compound denoted by general formula (5) above is produced by a microorganism which belongs to the genus Monascus and has the ability to produce monascus pigment.

It is an object of the present invention to provide the method described above, wherein the compound denoted by general formula (5) above is collected from the culture product of a microorganism which belongs to the genus Monascus and has the ability to produce a monascus pigment, and wherein said culture is conducted while feeding on acetic acid under acidic conditions.

It is an object of the present invention to provide the method described above, wherein said culture product is in the form of pigment-containing wet cell mass.

The present invention provides a chitosan oligosaccharide-bonded pigment with better stability in aqueous solutions and light stability than conventional monascus pigments by bonding a chitosan oligosaccharide with rubropunctatin and/or monascorubrin.

### BRIEF DESCRIPTION OF THE DRAWINGS

FIG. 1 shows a graph showing stability results for the pigment solutions of Example 4.

FIG. 2 shows a graph showing stability results for the pigment solutions of Example 5.

### DETAILED DESCRIPTION OF EXEMPLARY EMBODIMENTS

The present inventions are described in detail below.

The pigments produced by Monascus fungi have the structure denoted by general formula (5) below. In the formula, the compound is rubropunctatin when R is C₅H₁₁ and monascorubrin when R is C₇H₁₅. Hereinafter, rubropunctatin and monascorubrin may be collectively referred to simply as monascus pigments. 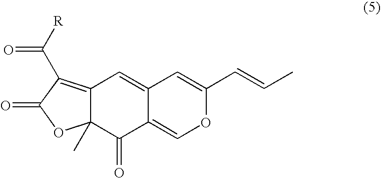
wherein R denotes C₅H₁₁ (rubropunctatin) or C₇H₁₅ (monascorubrin).

As shown in reaction equation (6) below, the compound of the present invention is produced in the form of a red pigment by reacting a monascus pigment with a chitosan oligosaccharide: 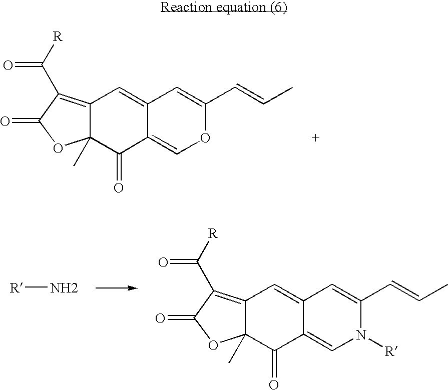
wherein R denotes C₅H₁₁ or C₇H₁₅ and R′ denotes a side chain moiety other than an amino group of the chitosan oligosaccharide.

The compound of the present invention, produced by the above-described reaction, has the structure given by general formula (1) below: 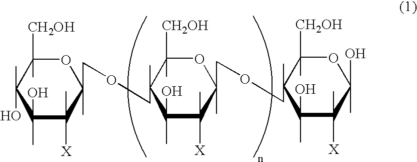
wherein n denotes an integer from 0 to 8 and wherein each X denotes either a residue denoted by general formula (2) below or an amino group and at least one X is a residue denoted by general formula (2) below; and 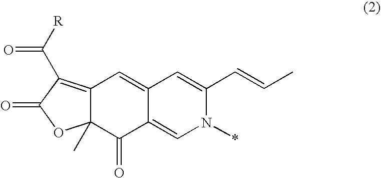
wherein R denotes C₅H₁₁ or C₇H₁₅

That is, the compound produced by reaction equation (6) comprises a pigment to which is bonded a chitosan oligosaccharide (chitosan oligosaccharide-bonded pigment). For example, when the chitosan oligosaccharide is a glucosamine dimer, the structure becomes that given by general formula (7) below during the reaction with the monascus pigment of formula (2) above; when a glucosamine trimer, the structure becomes that given by general formula (8) below; and when a tetramer, the structure becomes that given by general formula (9) below. As the degree of polymerization of the glucosamine increases, the chitosan oligosaccharide and monascus pigment bond according to this same pattern. Formulas (7), (8), and (9) below show structures obtained when a chitosan oligosaccharide and a monascus pigment bond 1-to-1. However, since the chitosan oligosaccharide that is supplied to the reaction has multiple amino groups, for example, compounds in which multiple monascus pigments are bonded to the chitosan oligosaccharide, as shown in formula (10) below, are also covered by the compound of the present invention. That is, when the degree of polymerization of the glucosamine is denoted by n, 1 to n of monascus pigments are bonded to each chitosan oligosaccharide. The chitosan oligosaccharide-bonded pigment compound of the present invention is a reaction product in the form of a single substance, or a mixture of two or more such substances. 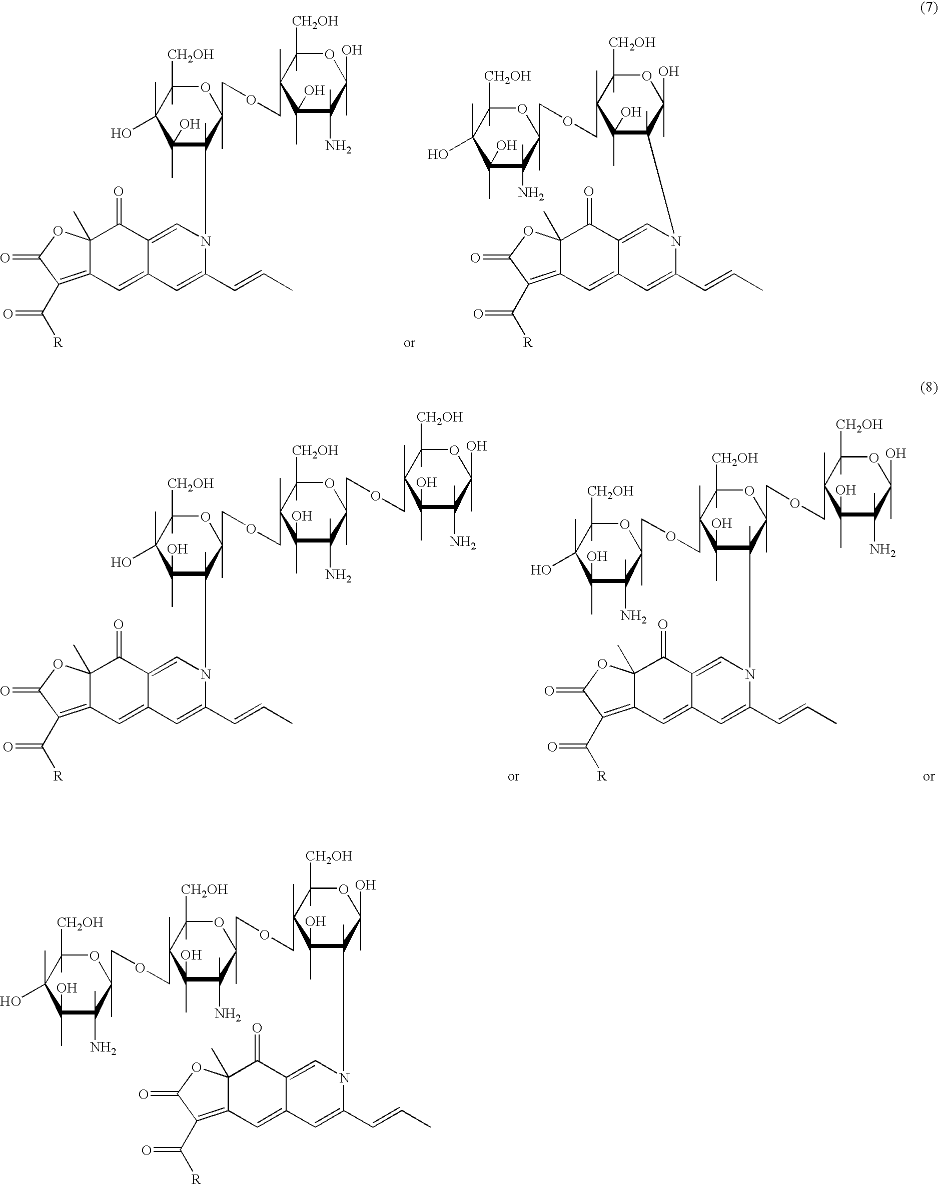 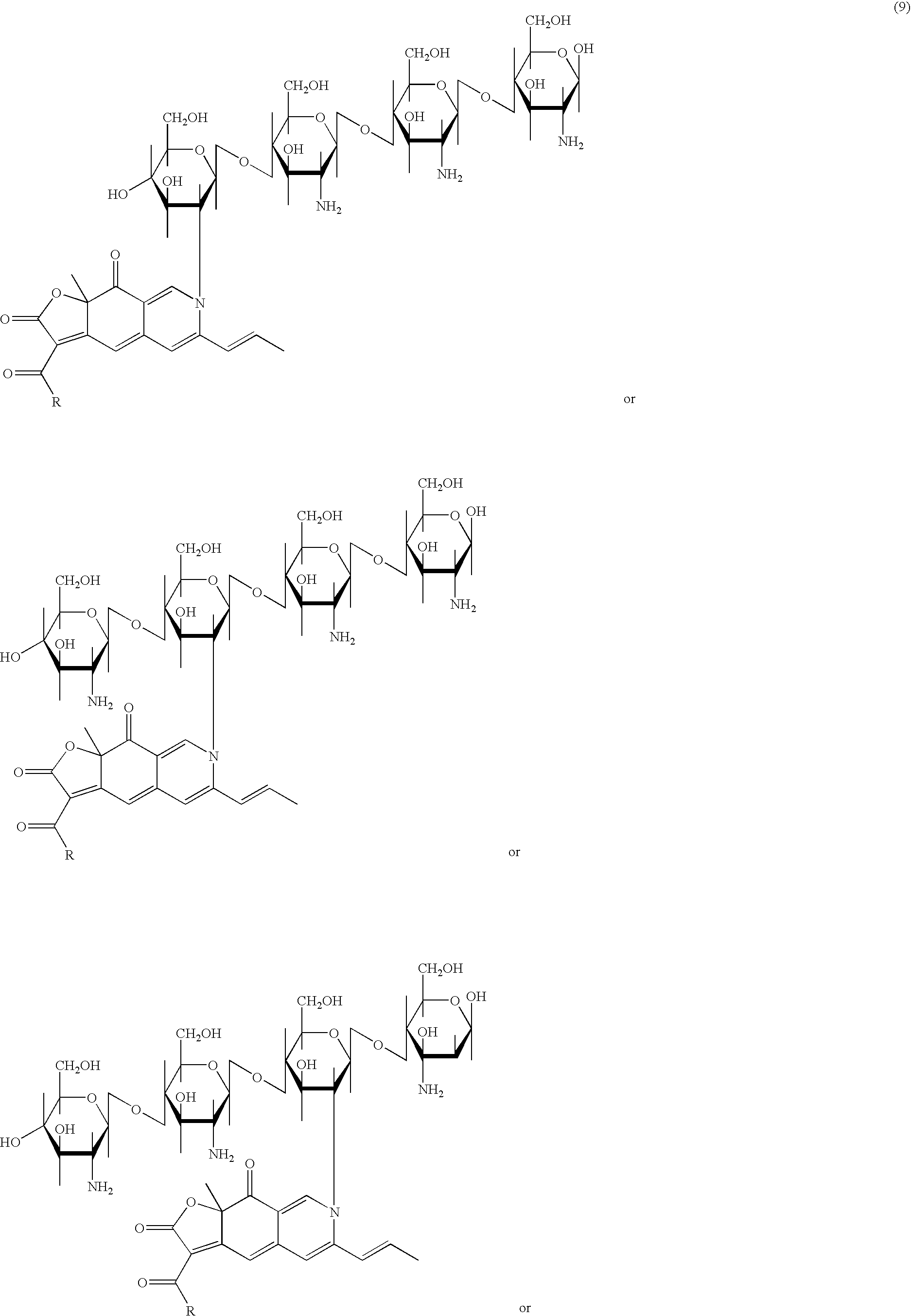 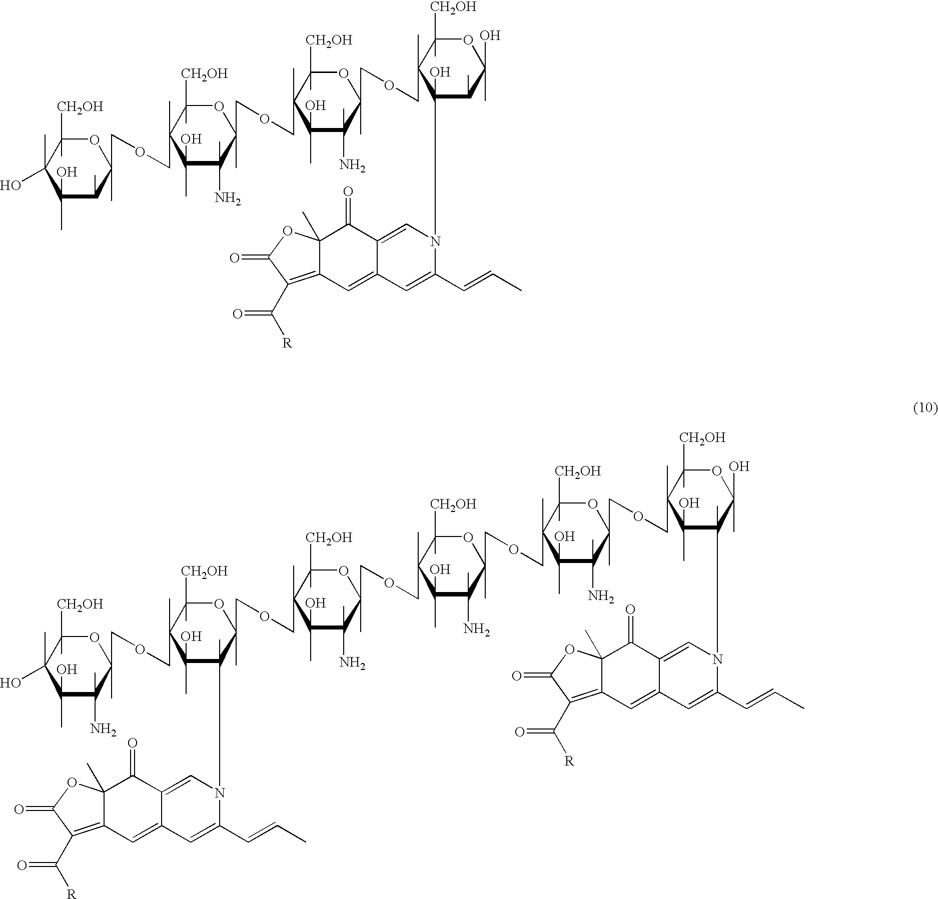
R denotes C₅H₁₁ or C₇H₁₅ in the formulas (7), (8), (9) and (10) above.

Chitosan oligosaccharides are industrially manufactured by hydrolysis of a chitosan which is a deacetylation product of the chitin contained in shells of crustaceans such as crabs and shrimp, with an acid or an enzyme. The hydrolysis product of chitosan is a mixture of various chitosan oligosaccharides of varying degrees of polymerization. A method such as column chromatography or solvent fractionation can be used to fractionate chitosan oligosaccharides of different degrees of polymerization. The chitosan oligosaccharide employed to manufacture the compound of the present invention may be in the form of a mixture, or may be purified form by fractionation; a degree of polymerization of 2 to 10 is preferred. Various commercially available chitosan oligosaccharides may be employed. For example, pure products such as “Chitosan Dimer to Hexamer” (made by Seikagaku Corporation) and mixed products such as “Oligoglucosamine” (tradename of product made by Koyo Chemical Co., Ltd.) and “Chimica Chitosan Oligosaccharide COS-A” (tradename of product made by Chimica K.K.) may be employed.

Monascus pigments are produced by various Monascus fungi, For example, powdered monascus pigment obtained from solid cultures of Monascus fungi, liquid pigment obtained from liquid cultures, and monascus ethanol extracts can be employed. The monascus fungus can be any fungus belonging to the genus Monascus. Examples are Monascus purpureus, Monascus anka, Monascus ruber; Monascus pilosus, and variants and mutants thereof.

Specifically, Monascus purpureus NBRC4478, Monascus purpureus ATCC16360, Monascus ruber NBRC9203, and Monascus pilosus NBRC4480 can be employed.

NBRC4478, 9203, and 4480 are available from the Biological Resource Center, Department of Biotechnology, National Institute of Technology and Evaluation (NITE), an Independent Administrative Institution (2-5-8, Kazusakamatari, Kisarazu-shi, Chiba-ken, Postal Code 292-0818).

ATCC16360 is available from the American Type Culture Collection (ATCC) (ATCC, P.O. Box 1549, Manassas, Va. 20108, USA).

The method used to culture the Monascus fungus is not specifically limited and may be a known method. Preferably, the Monascus fungus is cultured by a method permitting the accumulation of a high concentration of rubropunctatin or monascorubrin, which is then reacted with a chitosan oligosaccharide. An example of a culture method that accumulates high concentrations of rubropunctatin and monascorubrin is one in which the pH is controlled by the addition of acetic acid (Japanese Patent Application Publication No. 2003-268254), Extraction of the rubropunctatin or monascorubrin that has accumulated in the cell mass and the reaction bonding the pigment to a chitosan oligosaccharide can be conducted simultaneously following culturing. Alternatively, the pigment can be extracted first and then subjected to a bonding reaction with a chitosan oligosaccharide. For the sake of convenience, an organic solvent and a chitosan oligosaccharide solution are desirably added to a cell mass containing the pigment to conduct extraction and the bonding reaction. From the perspective of purity, rubropunctatin or monascorubrin that has been accumulated to a high concentration in a medium is first extracted with an organic solvent and then subjected to a bonding reaction with a chitosan oligosaccharide. In either case, the chitosan oligosaccharide is desirably added in a ratio ranging from equivalence to 500 times equivalence, preferably equivalence to about 50 times equivalence, based on the ratio of the glucosamine of the chitosan oligosaccharide to the number of moles of rubropunctatin or monascorubrin. In the latter case, the extracted rubropunctatin or monascorubrin can be dissolved in methanol, ethanol, or the like and a chitosan oligosaccharide aqueous solution added. The concentration of the pigment mixture is not specifically limited; it suffices for the content to fall within a range permitting dissolution of the pigment in the reaction solution.

The chitosan oligosaccharide aqueous solution that is added is adjusted to pH 6 to 10, preferably pH 6 to 8. This is because the bonding reaction between the chitosan oligosaccharide and the pigment tends not to progress unless the pH is close to neutral or on the alkaline side (Journal of Industrial Microbiology, Vol. 16, pp. 163-170 (1996): Journal of Industrial Microbiology, Vol. 16, pp 163-170 (1996)). The chitosan oligosaccharide may be dissolved in a buffer solution instead of an aqueous solution for use in the reaction. In that case, the buffer solution is not specifically limited other than that it does not contribute to the reaction, As an example, a phosphate buffer solution or McIlvaine buffer solution may be employed. The temperature is from room temperature to 80° C. and stirring is conducted for 1 to 72 hours. Due to poor solubility in water, rubropunctatin or monascorubrin is filtered together with the cell mass and recovered by centrifugation in the above-mentioned culturing in which the pH is regulated by adding acetic acid (Japanese Patent Application Publication No. 2003-268254). Ethanol and a chitosan oligosaccharide aqueous solution may be added directly to the mixture of cell mass and pigment to produce chitosan oligosaccharide-bonded pigment. In that case, the pigment extraction step is omitted.

A chitosan oligosaccharide may be added during culturing to obtain chitosan oligosaccharide-bonded pigment, a water-soluble red pigment. The quantity of chitosan oligosaccharide that is added to the medium is not specifically limited. Normally, for a liquid medium, a quantity of 0.05 weight percent or more, preferably 0.5 to 5 weight percent, is added.

The red pigment of the present invention can be used in the form of the unaltered reaction solution; filtered and centrifuged to remove insoluble matter; purified with resin or the like as needed; or concentrated and dried to obtain a red pigment composition for use. When preparing the chitosan oligosaccharide-bonded pigment of the present invention as a pigment composition for use in foods, in the same manner as for known monascus pigments, sugars such as lactose, D-mannitol, and D-sorbitol; starches such as cornstarch and potato starch; inorganic salts such as calcium phosphate and calcium sulfate; and other excipients, diluents, and additives may be suitably formulated as needed. Based on the intended use, other colorants may be admixed. When being employed as a printing ink, for example, the pigment can be prepared as the medium for ink (aqueous or oil-based ink medium) described in International Patent Application Publication WO02/088265, or as a pigment composition to which dispersing agents and binders are added.

Examples of aqueous ink solvents are: alcohols such as methanol, ethanol, propanol, isopropanol, butanol, isobutanol, sec-butanol, t-butanol, pentanol, hexanol, cyclohexanol, and benzyl alcohol; polyhydric alcohols such as ethylene glycol, diethylene glycol, triethylene glycol, polyethylene glycol, propylene glycol, dipropylene glycol, polypropylene glycol, butylene glycol, hexanediol, pentanediol, glycerin, hexanetriol, and thiodiglycol; glycol derivatives such as ethylene glycol monomethyl ether, ethylene glycol monoethyl ethyl, ethylene glycol monobutyl ether, diethylene glycol monoethyl ether, diethylene glycol monobutyl ether, propylene glycol monomethyl ether, propylene glycol monobutyl ether, dipropylene Glycol monomethyl ether, triethylene glycol monomethyl ether, ethylene glycol diacetate, ethylene glycol monomethyl ether acetate, triethylene glycol monomethyl ether, triethylene glycol monoethyl ether, and ethylene glycol monophenyl ether; amines such as ethanol amine, diethanol amine, triethanol amine, N-methyl diethanol amine, N-ethyl diethanol amine, morpholine, N-ethyl morpholine, ethylene diamine, diethylene triamine, triethylene tetramine, polyethylene imine, and tetramethyl propylene diamine; and polar solvents such as formamide, N,N-dimethyl formamide, N,N-dimethyl acetamide, dimethyl sulfoxide, sulfolane, 2-pyrrolidone, N-methyl-2-pyrrolidone, N-vinyl-2-pyrrolidone, 2-oxazolidone, 1,3-dimethyl-2-imidazolidinone, acetonitrile, and acetone. Preferred aqueous ink solvents are water, methanol, ethanol, propanol, butanol, and diethylene glycol. These solvents may be employed singly or in mixtures of two or more. When mixing a water-soluble organic solvent with water for use, the concentration of the water-soluble organic solvent in the aqueous solution is desirably 80 weight percent or less.

An oil-based ink solvent can be suitably selected as desired from among the usual organic solvents Examples of preferred oil-based ink solvents are: alcohols such as ethanol, pentanol, heptanol, octanol, cyclohexanol, benzyl alcohol, phenyl ethyl alcohol, phenyl propanol, furfuryl alcohol, and anise alcohol; glycol derivatives such as ethylene glycol monoethyl ether, ethylene glycol monophenyl ether, diethylene glycol monoethyl ether; diethylene glycol monobutyl ether, propylene glycol monoethyl ether, propylene glycol monophenyl ether, dipropylene glycol monomethyl ether, dipropylene glycol monoethyl ether, triethylene glycol monoethyl ether, ethylene glycol diacetate, ethylene glycol monoethyl ether acetate, and propylene glycol diacetate; ketones such as benzyl methyl ketone, diacetone alcohol, and cyclohexanone; ethers such as butyl phenyl ether, benzyl ethyl ether, and hexyl ether; esters such as ethyl acetate, amyl acetate, benzyl acetate, phenyl ethyl acetate, phenoxyethyl acetate, ethyl phenyl acetate, benzyl propionate, ethyl benzoate, butyl benzoate, ethyl laurate, butyl laurate, tributyl phosphate, diethyl phthalate, dibutyl phthalate, diethyl malonate, dipropyl malonate, diethyl diethylmalonate, dibutyl adipate, di(2-methoxyethyl)adipate, diethyl sebacate, diethyl maleate, dibutyl maleate, dioctyl maleate, diethyl fumarate, dioctyl fumarate, and 3-hexenyl cinnamate; hydrocarbon solvents such as petroleum ether, petroleum benzyl, tetraline, decaline, 1-amylbenzene, and dimethyl naphthaline; and polar solvents such as acetonitrile, formamide, N,N-dimethyl formamide, N,N-dimethyl acetamide, dimethyl sulfoxide, sulfolane, propylene carbonate, N-methyl-2-pyrrolidone, N-vinyl-2-pyrrolidone, and N,N-diethyl dodecaneamide. These solvents may be employed singly or in mixtures of two or more. The chitosan oligosaccharide-bonded pigment of the present invention may be dissolved in one or more of the above organic solvents, or dispersed in a suitable dispersing agent. For water-based and oil-based inks, the viscosity is desirably adjusted to 40 mPa·s or less and the surface tension is desirably adjusted to 20 to 100 mN/m.

The solvent for a solid ink is suitably selected for use from among phase-changing media that are solid but become liquid when heated. Examples are: natural waxes such as beeswax, carnauba wax, rice wax, Japan wax, jojoba oil, spermaceti, candelilla wax, lanolin, montan wax, ozokerite, ceresin, paraffin wax, microcrystalline wax, and petrolactam; organic acids such as polyethylene wax, chlorinated hydrocarbons, palmitic acid, stearic acid, behenic acid, tiglic acid, 2-acetonaphthobehenic acid, 1,2-hydroxystearic acid, and dihydroxystearic acid; alcohols such as dodecanol, tetradecanol, hexadecanol, eicosanol, docosanol, tetracosanol, hexacosanol, octacosanol, dodecenol, myricyl alcohol, tetracenol, hexadecenol, eicosenol, docosenol, pinene glycol, hinokiol, butinediol, nonanediol, isophthalic alcohol, methyserine, hexanediol, decanediol, tetradecanediol, hexadecanediol, docosanediol, tetracosanediol, terepineol, phenyl glycerine, eicosanediol, octanediol, and phenyl propylene glycol; phenols such as bisphenol A and p-α-cumyl phenol; organic acid esters of the above-listed organic acids and glycerine, ethylene glycol, and diethylene glycol; cholesterol fatty acid esters such as cholesterol stearate, cholesterol palmitate, cholesterol myristate, cholesterol behenate, cholesterol laurate, and cholesterol melissate; sugar fatty acid esters such as saccharose stearate, saccharose palmitate, saccharose behenate, saccharose laurate, saccharose melissate, glucose stearate, lactose palmitate, lactose behenate, lactose laurate, and lactose melissate; ketones such as benzoyl acetone, diacetobenzene, benzophenone, tricosanone, heptacosanone, heptatriacontanone, hentriacontanone, stearone, and laurone; amides such as oleamide, lauramide, stearamide, lysinamide, palmitamide, tetrahydrofuramide, erucamide, myristamide, 1,2-hydroxystearamide, N-stearyl erucamide, N-oleyl stearamide, N,N-ethylene bislauramide, N,N-ethylene bisstearamide, N,N-ethylene bisbehenamide, N,N-xylylene bisstearamide, N,N-butylene bisstearamide, N,N-dioleyl adipamide, N,N-dioleyl sebacamide, N,N-distearyl sebacamide, N,N-distearyl terephthalamide, phenacetin, toluamide, and acetamide; and sulfonamides such as p-toluene sulfonamide, ethyl benzene sulfonamide, and butyl benzene sulfonamide.

The chitosan oligosaccharide-bonded pigment of the present invention may be heated to a liquid state and dispersed or dissolved in the above-described solid medium, or may be dispersed or dissolved in combination with a dispersing agent or binder. The phase-change temperature of the solid medium is desirably 60 to 200° C., preferably 80 to 150° C.

A surfactant can be employed as dispersing agent. Cationic, anionic, amphoteric, and nonionic surfactants may all be employed. Examples of cationic surfactants are: aliphatic amine salts, aliphatic quaternary ammonium salts, benzalconium salt, benzetonium chloride, pyridinium salt, and imidazolium salt, Examples of anionic surfactants are: fatty acid soaps, N-acyl-N-methylglycine salt, N-acyl-N-methyl-β-alanine salt, N-acyl glutamates, acylated peptides, alkyl sulfonates, alkyl benzene sulfonates, alkyl naphthalene sulfonates, dialkyl sulfosuccinic ester salts, alkyl sulfoacetate, α-olefin sulfonates, N-acryl methyl taurine, sulfated oils, higher alcohol sulfuric ester salts, secondary higher alcohol sulfuric ester salts, alkyl ether sulfonates, secondary higher alcohol ethoxysulfates, fatty acid alkylolamide sulfuric ester salts, alkyl ether phosphoric ester salts, and alkyl phosphoric acid ester salts. Examples of amphoteric surfactants are: carboxybetain and sulphobetain-type surfactants, aminocarbonates, and imidazolinium betain. Examples of nonionic surfactants are polyoxyethylene secondary alcohol ether, polyoxyethylene alkyl phenyl ether, polyoxyethylene sterol ether, polyoxyethylene lanolin-derived polyoxyethylene polyoxypropylene alkyl ether, polyoxyethylene glycerin fatty acid esters, polyoxyethylene castor oil, polyoxyethylene sorbitol fatty acid esters, polyethylene glycol fatty acid esters, fatty acid monoglycerides, polyglycerin fatty acid esters, sorbitan fatty acid esters, propylene glycol fatty acid esters, sucrose fatty acid esters, fatty acid alkanolamides, polyoxyethylene fatty acid amides, polyoxyethylene alkyl amines, alkyl amine oxides, acetylene glycol, and acetylene alcohols.

Examples of binders are: water-soluble polymers such as gelatins, casein, gum arabic, sodium alginate, carbomethyl cellulose, polyvinyl alcohol, polyvinyl pyrrolidone, sodium polyacrylate, and polyacrylamide; synthetic resin latexes such as synthetic rubber latex; and organic solvent-soluble resins such as polyvinyl butyral, polyvinyl chloride, vinyl polyacetate, polyacrylonitrile, polymethyl methacrylate, polyvinyl formal, melanine resin, polyamide resin, phenol resin, polyurethane resin, and alkyd resin.

Various other additives may be added as needed Examples of such additives are pH-adjusting agents, viscosity-adjusting agents, penetrants, surface tension adjusting agents, antioxidants, preservatives, and antifungals.

It is important that the chitosan oligosaccharide-bonded pigment of the present invention be a pigment obtained by a bonding reaction of rubropunctatin and monascorubrin with a chitosan oligosaccharide. For example, Japanese Patent Application Publication No Sho 62-297365 discloses a pigment stabilization method in which chitosan is added to a pigment. In the bonding reaction of rubropunctatin, monascorubrin, and a group having an amino group, a Schiff's base is formed. The reaction progresses at from close to neutral to alkaline pH; it will not take place at acidic pH (Journal of Industrial Microbiology, Vol. 16, pp 163-170 (1996); Journal of Industrial Microbiology, Vol. 16, pp 163-170 (1996)). In the above-cited patent (Japanese Patent Application Publication No Sho 62-297365), the chitosan is dissolved in a slightly acidic solution and is added to a commercial monascus pigment; no bonding reaction takes place, Further, the commercial water-soluble monascus pigments rubropunctatin and monascorubrin are nearly always already bonded to a compound having an amino group. Since the bonding reaction is an irreversible reaction, the content of rubropunctatin and monascorubrin is low. Accordingly, even when chitosan is added to a commercial monascus pigment, no chitosan-bonded pigment forms; product obtained by adding chitosan oligosaccharide to a commercial monascus pigment (in which the two are present without bonding) differs from the pigment of the present invention in which a chitosan oligosaccharide is bonded to a monascus pigment.

The red pigment composition of the present invention can be employed in a variety of applications, including as a colorant in foods and as a printing-use ink material.

The present invention will be described in detail below through Examples.

### EXAMPLES

### Manufacturing Example 1

(Preparation of a Pigment Composition Containing Rubropunctatin and Monascorubrin)

YM medium (1 weight percent glucose, 0.3 weight percent yeast extract (made by Difco Laboratories, Inc.), 0.3 weight percent malto extract (made by Difco Laboratories, Inc.), and 0.5 weight percent bactopeptone (made by Difco Laboratories, Inc.)) was adjusted to pH 6.5. 1 L thereof was charged to 5 L Sakaguchi flasks, and autoclaved for 20 minutes at 120° C. After cooling, one platinum loop of Monascus fungus (Monascus purpureus (NBRC4478)) that had been cultured on a slant surface on YM agar medium was inoculated. The fungus was cultured with shaking for two days at 30° C., yielding a seed fungus solution.

450 mL of YM medium identical to the above was charged to a one-liter glass jar and autoclaved for 20 minutes at 120° C. After cooling, the medium was seeded with 10 percent (v/v) of the above seed fungus solution. While employing a pH adjusting agent in the form of acetic acid to maintain the culture solution at pH 4.0 from the start of culturing, the fungus was cultured with stirring and ventilation for 7 days at 30° C. After the main culture had been completed, the culture solution was placed on a centrifugal separator (9,000 rpm, 10 minutes) and separated into supernatant and a cell mass, yielding a pigment-containing wet cell mass. This was freeze dried; the moisture content was measured to be 75.6 weight percent.

Ten liters of ethyl acetate were added to 400 g of the wet cell mass. Following stirring for 1 hour, the cell mass and filtrate were separated out with filter paper. The aqueous layer was removed from the filtrate, yielding an ethyl acetate layer. The ethyl acetate layer was washed with an equal quantity of water. This operation was repeated twice. Following washing, the ethyl acetate extract was concentrated and dried, yielding a reddish-orange pigment containing rubropunctatin and monascorubrin.

### Manufacturing Example 2

(Preparation of Rubropunctatin and Monascorubrin)

When analyzed by high-performance liquid chromatography, the pigment composition prepared in Manufacturing Example 1 exhibited four major peaks at a detection wavelength of 400 nm. The peaks with retention times of 7.1 minutes and 11.6 minutes were collected and purified. Visible and UV absorbance spectra, mass analysis, and NMR measurement were conducted, identifying the peaks as rubropunctatin and monascorubrin. The high-performance liquid chromatography analysis conditions were as follows.

Column: CAPCELL Pak C18 UG120, φ4.6 mm×250 mm (made by Shiseido, Inc.)

Mobile phase: water/acetonitrile (30/70)

Flow rate: 1 mL/minute

Temperature: room temperature

Detection: 400 nm

### Example 1

Chitosan dimer (made by Seikagaku Corporation) was dissolved in water to 3.4 mg/L. The aqueous solution was adjusted to pH 7 and mixed 1:1 with a 240 μg/L methanol solution of the rubropunctatin prepared in Manufacturing Example 2. The mixture was reacted overnight with stirring. The color of the reaction solution changed from orange to deep red. When analyzed by high-performance liquid chromatography under the conditions given in Manufacturing Example 2, the rubropunctatin peak was not detected. When the composition of the mobile phase in the analysis conditions of the high-performance liquid chromatography of Manufacturing Example 2 was changed to 0.05 percent TFA/0.05% TFA-containing acetonitrile (70/30) and detection was conducted at 500 nm, three major peaks appeared. The molecular weight of all three of these peaks as measured by LC-MS was 676. This was equal to the sum of the molecular weight of rubropunctatin (354) and the molecular weight of chitosan dimer (340) less the molecular weight of water (18), and was presumed to be the structure shown in general formula (11).

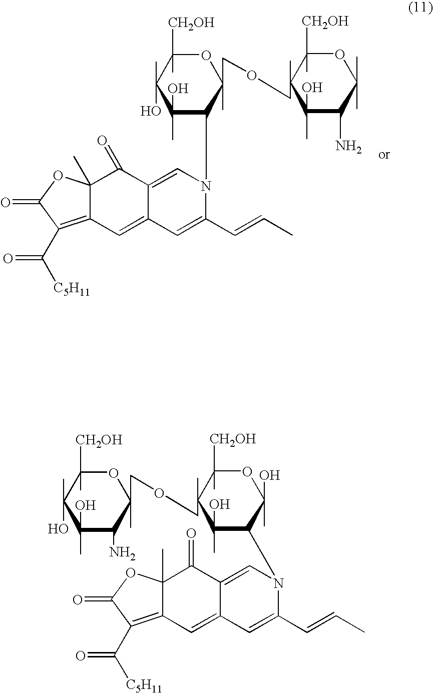

### Example 2

Chitosan tetramer (made by Seikagaku Corporation) was dissolved to 3.2 mg/mL in water. The solution was reacted with rubropunctatin in the same manner as in Example 1. The color of the reaction solution changed from orange to deep red. High-performance liquid chromatographic analysis was conducted under the conditions given in Manufacturing Example 2, but the rubropunctatin peak was not detected. When the composition of the mobile phase in the analysis conditions of the high-performance liquid chromatography of Manufacturing Example 2 was changed to 0.05 percent TFA/0.05% TFA-containing acetonitrile (70/30) and detection was conducted at 500 nm, four major peaks appeared. The molecular weight of all four of these peaks as measured by LC-MS was 998. This was equal to the sum of the molecular weight of rubropunctatin (354) and the molecular weight of chitosan tetramer (662) less the molecular weight of water (18), and was presumed to be the structure shown in general formula (12).

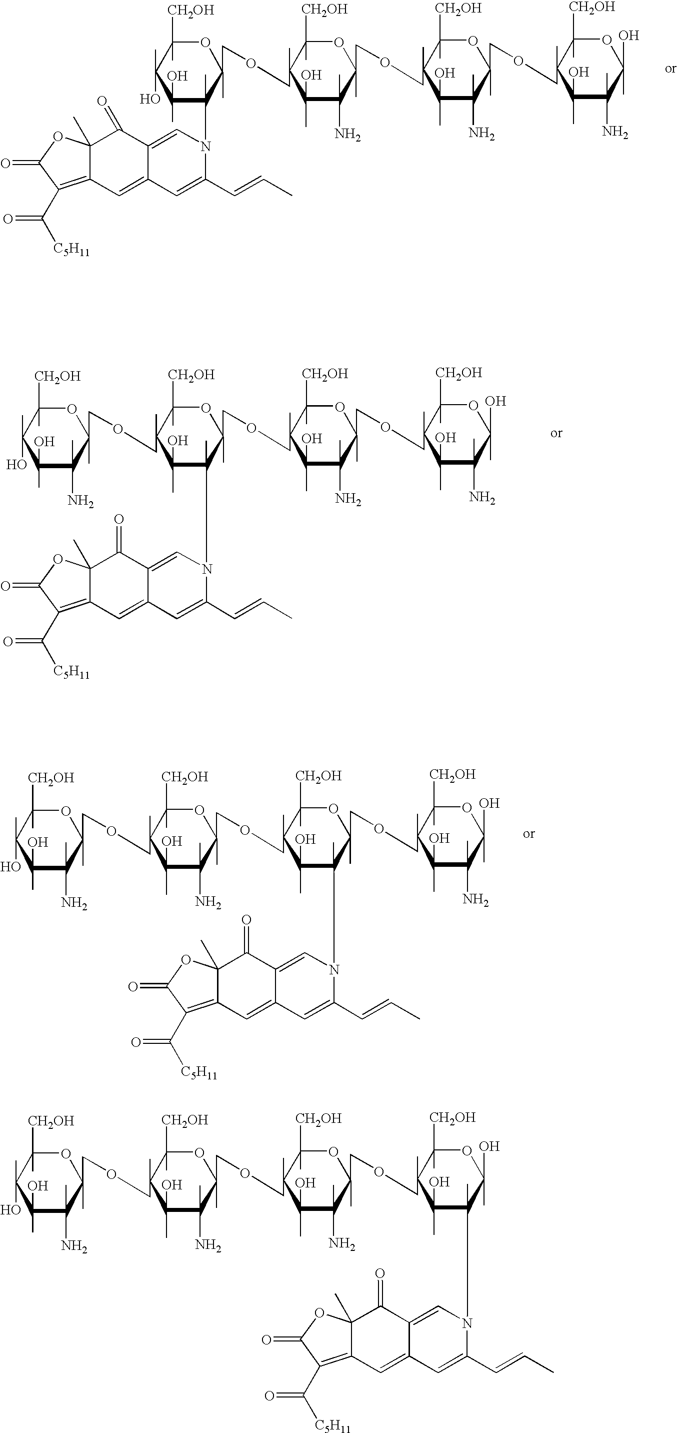

### Example 3

Glucosamine, chitosan dimer, chitosan tetramer, chitosan hexamer (made by Seikagaku Corporation) were dissolved in water to a concentration of 16 mM based on glucosamine and adjusted to pH 7 with sodium hydroxide aqueous solution. For comparison, a 6 mM aqueous solution of monosodium glutamate was also prepared. When these solutions were separately mixed 1:1 with the 40 μg/mL methanol solution of monascorubrin prepared in Manufacturing Example 2 and reacted overnight with stirring. The color of the reaction solution changed from orange to deep red. When analyzed by high-performance liquid chromatography under the conditions given in Manufacturing Example 2, no monascorubrin peak was detected from any of the reaction solutions. The pigment was presumed to have changed into glucosamine and chitosan oligosaccharide bonded pigment and glutamic acid-bonded pigment. The solvent in the solution was dried with a centrifugal concentrator. The concentration was adjusted by adding water to 0.55 of absorbance at 500 nm for a 1/10 diluted solution. Following adjustment to pH 7, the solution was charged to a threaded-neck glass bottle and stored in a dark location for 15 hours at 60° C. The 500 nm absorbance was measured before and after storage and the ratio of the absorbance following storage to the absorbance prior to storage was calculated as the residual rate (%). The stability rates were compared. As a result, as shown in Table 1, the stability in water of chitosan oligosaccharide-bonded pigment was better than that of glutamic acid-bonded pigment and that of glucosamine-bonded pigment. The higher the degree of polymerization of glucosamine, the greater the degree of stability afforded.

TABLE 1

| | residual rate (%) |
|---|---|
| glucosamine-bonded pigment | 28.7 |
| chitosan dimer-bonded pigment | 48.8 |
| chitosan tetramer-bonded pigment | 64.7 |
| chitosan hexamer-bonded pigment | 73.4 |
| glutamic acid-bonded pigment | 29.7 |

### Example 4

“Oligoglucosamine” (tradename of product made by Koyo Chemical Con, Ltd.) was dissolved to a concentration of 34.4 mg/mL in water and the solution was adjusted to pH 7 with sodium hydroxide aqueous solution. A 24 g quantity of the pigment-containing wet cell mass cultured and prepared in Manufacturing Example 1 was added to 608 mL of this solution. Then, 608 ml of ethanol was added to this solution. The solution was then reacted overnight with stirring at room temperature. The color of the solution went from orange to deep red. The reaction solution was filtered with filter paper (No. 5B, made by Kiriyama Glass Works Co., Ltd.), the cell mass was removed, and the ethanol was removed from the filtrate with an evaporator. This solution was filtered again to remove water-insoluble components, after which the filtrate was freeze dried, yielding 21.7 g of red pigment powder. The pigment powder was dissolved to a concentration of 20 mg/mL in 50 percent ethanol and analyzed by high-performance liquid chromatography under the same conditions as in Manufacturing Example 2. No peaks were detected for either rubropunctatin or monascorubrin. The original pigment was presumed to have been converted to a pigment containing a principal component in the form of chitosan oligosaccharide-bonded pigment.

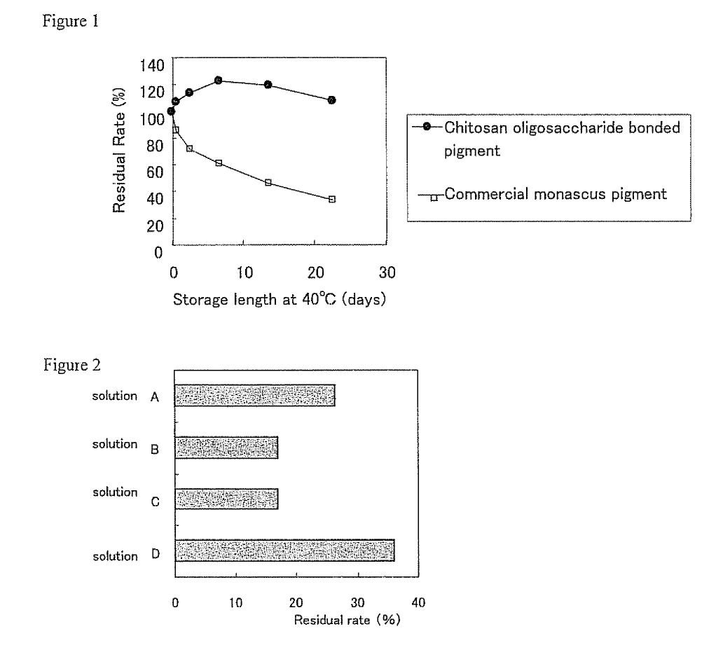

The chitosan oligosaccharide-bonded pigment powder obtained was dissolved in water to a concentration of 100 mg/mL and adjusted to pH 7. The absorbance at 500 nm was then measured. Commercial monascus pigment (“Benikoji Pigment” made by Kanto Chemical Co., Inc.) was dissolved in water to the same absorbance as the chitosan oligosaccharide-bonded pigment and adjusted to pH 7. These solutions were then separately charged to threaded-neck glass bottles and stored in a dark location at 40° C. The absorbance at 500 nm was measured before and after storage. The ratio of the absorbance following storage to the absorbance prior to storage was calculated as the residual rate (%) and the stability rates were compared. As a result, as shown in FIG. 1, the chitosan oligosaccharide-bonded pigment exhibited better stability in aqueous solution than the commercial monascus pigment.

### Example 5

Chitosan (“Koyo Chitosan DAC-100” made by Koyo Chemical Co., Ltd.) and chitosan oligosaccharide (“Oligoglucosamine” made by Koyo Chemical Co., Ltd.) were each dissolved in 0.05 M acetic acid aqueous solutions to a concentration of 2 mg/mL. A 2 mg/mL quantity of commercial monascus pigment (“Benikoji Pigment” made by Kanto Chemical Co., Ltd.) was then added to the chitosan/acetic acid aqueous solution, chitosan oligosaccharide/acetic acid aqueous solution, and a 0.05 M acetic acid aqueous solution. Following stirring, the precipitate was removed by centrifugation and the solutions (Solutions A, B, and C, respectively) were adjusted with 0.05 M acetic acid to 0.6 of absorbance at 500 nm when diluted by 1/3. The chitosan oligosacclharide-bonded pigment prepared in Example 4 was dissolved in 0.05 M acetic acid and the concentration was adjusted to the above-stated absorbance (Solution D). Since chitosan is insoluble in water, a solution of chitosan in acetic acid was employed in an attempt to prepare chitosan-bonded pigment by the method of Example 4. However, the reaction did not progress and no water-soluble bonded pigment was obtained. Table 2 shows the compositions of these pigment solutions. The state of the monascus pigment was as follows: Solution A: monascus pigment in the presence of chitosan; Solution B: monascus pigment in the presence of chitosan oligosaccharide; Solution C: monascus pigment alone and Solution D: monascus pigment bonded to chitosan oligosaccharide.

TABLE 2

| | solution | Pigment solution composition |
|---|---|---|
| Solution A | monascus pigment in the | Chitosan/monascus pigment/ |
| | presence of chitosan | acetic acid |
| Solution B | monascus pigment in the | Chitosan oligosaccharide/ |
| | presence of chitosan | monascus pigment/acetic acid |
| | oligosaccharide | |
| Solution C | monascus pigment alone | monascus pigment/acetic acid |
| Solution D | monascus pigment bonded to | monascus pigment bonded to |
| | chitosan oligosaccharide | chitosan oligosaccharide/ |
| | | acetic acid |

Solutions A to D in Table 2 were charged to threaded-neck bottles and stored in a dark location for 15 hours at 40° C. The 500 nm absorbance was measured before and after storage and the ratio of the absorbance following storage to the absorbance prior to storage was calculated as the residual rate (%). The stability rates were compared. As a result, as shown in FIG. 2, the monascus pigment did not exhibit a marked stabilization effect when in the presence of chitosan or chitosan oligosaccharide, but did exhibit a marked stabilization effect when bonded to chitosan oligosaccharide.

### Example 6

The chitosan oligosaccharide-bonded pigment prepared in Example 4 and commercial monascus pigment (“Benikoji Pigment” made by Kanto Chemical Co., Ltd.) were separately dissolved to a concentration of 20 mg/mL in water and adjusted to pH 7. The solutions were charged to glass bottles and irradiated for three days by a 2,800 lux fluorescent lamp at 25° C. Identical samples were placed in a dark location. The 500 nm absorbance was measured before and after storage and the light irradiation residual rate and dark storage residual rate were calculated. The E value (%) was calculated as (residual rate of sample irradiated with light)/(residual rate of sample stored in dark)×100 and used as an index for comparison of light stability. As a result, the E value (%) of the chitosan oligosaccharide-bonded pigment was 56.0 percent and that of commercial monascus pigment was 41.5 percent. The chitosan oligosaccharide-bonded pigment was found to have a stability for irradiation with light that was higher than that of common monascus pigment.

As set forth above, the present invention provides a monascus pigment affording better stability in aqueous solutions and better light stability than conventional monascus pigment by bonding rubropunctatin and monascorubrin to a chitosan oligosaccharide.

Although the present invention has been described with reference to the preferred examples, it should be understood that various modifications and variations can be easily made by those skilled in the art without departing from the spirit of the invention. Accordingly, the foregoing disclosure should be interpreted as illustrative only and is not to be interpreted in a limiting sense. The present invention is limited only by the scope of the following claims along with their full scope of equivalents. Each of the aforementioned documents, including the foreign priority document, is incorporated by reference herein in its entirety.

We claim:

1. A compound denoted by general formula (1) below; 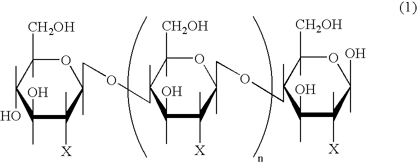
wherein n denotes an integer from 0 to 8 and wherein each X denotes either a residue denoted by general formula (2) below or an amino group and at least one X is a residue denoted by general formula (2) below; and 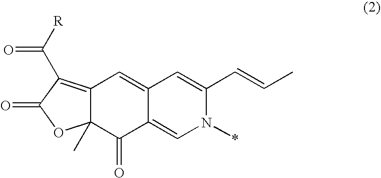
wherein R denotes C₅H₁₁ or C₇H₁₅

2. The compound according to claim 1, which is denoted by general formula (3) below; 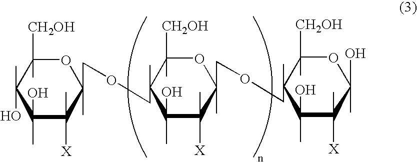
wherein n denotes an integer from 0 to 8 and wherein one X is a residue denoted by general formula (2) below and the others are amino groups; and 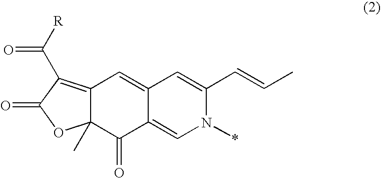
wherein R denotes C₅H₁₁ or C₇H₁₅.

3. A water-soluble composition comprising a mixture of one or more of the compounds according to claim 1.

4. A pigment comprising one or more of the compounds according to claim 1.

5. A pigment composition comprising a mixture of one or more of the compounds according to claim 1 and an ink-use solvent.

6. A method for manufacturing the compound of claim 1 comprising at least the steps of:
supplying a chitosan oligosaccharide denoted by general formula (4) below and a compound denoted by general formula (5) below to the transamination reaction denoted by reaction equation (6) below at pH of between 6 and 10; and
replacing the oxygen atom of a pyrane ring in the compound denoted by general formula (5) below with the nitrogen atom of an amino group on at least one structural sugar in the oligosaccharide; 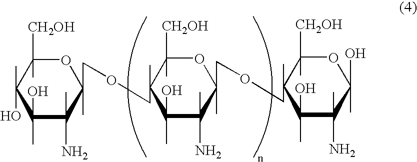
wherein n denotes an integer from 0 to 8; 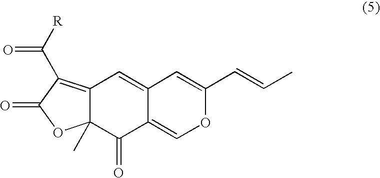
wherein R denotes C₅H₁₁ or C₇H₁₅. 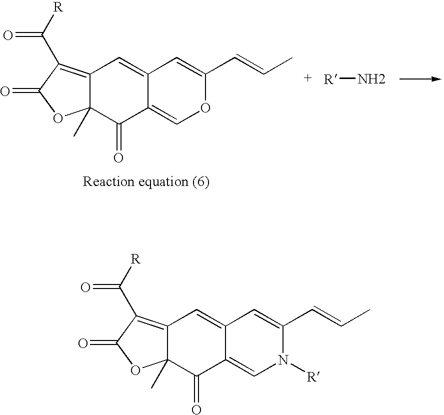
wherein R denotes C₅H₁₁ or C₇H₁₅ and R′ denotes a side chain moiety other than an amino group of the chitosan oligosaccharide.

7. The method according to claim 6, wherein the compound denoted by general formula (5) above is produced by a microorganism which belongs to the genus Monascus and has the ability to produce monascus pigment.

8. The method according to claim 7, wherein the compound denoted by general formula (5) above is collected from the culture product of a microorganism which belongs to the genus Monascus and has the ability to produce a monascus pigment, and wherein said culture is conducted while feeding on acetic acid under acidic conditions.

9. The method according to claim 7, wherein said culture product is in the form of pigment-containing wet cell mass.
